(19) United States
(12) Patent Application Publication
(10) Pub. No.: US 2004/0002766 A1
(43) Pub. Date: Jan. 1, 2004

(54) Prosthetic devices having diffusion-hardened surfaces and bioceramic coatings

(76) Inventors: Gordon Hunter, Memphis, TN (US); Harsh Gupta, Memphis, TN (US); Daniel Heuer, Memphis, TN (US); Marc Long, Memphis, TN (US); James Davidson, Germantown, TN (US); Ajit Mishra, San Antonio, TX (US)

Correspondence Address: SMITH & NEPHEW, INC., 1450 E. BROOKS ROAD, MEMPHIS, TN, 38116, US

(21) Appl. No.: 10/185,386

(22) Filed: Jun. 27, 2002

(51) Int. Cl. A61F 2/30

(52) U.S. Cl. 623/20.21; 623/22.17; 623/23.53; 623/23.56

(57) ABSTRACT

A prosthetic device having at least part of its surface comprising a diffusion-hardened, in-situ formed oxidation or nitridation layer and with at least another part of its surface comprising a coating of bioceramic, preferably hydroxyapatite. The bone in-growth and on-growth promoting bioceramic further works synergistically with the diffusion-hardened surface in realizing a longer service life prosthetic.

### BACKGROUND OF THE INVENTION

Orthopedic implant materials must combine high strength, corrosion resistance and tissue compatibility. The longevity of the implant is of prime importance especially if the recipient of the implant is relatively young because it is desirable that the implant function for the complete lifetime of a patient. Because certain metal alloys have the required mechanical strength and biocompatibility, they are ideal candidates for the fabrication of prostheses. These alloys include 316L stainless steel, chrome-cobalt-molybdenum alloys and, more recently, titanium alloys which have proven to be the most suitable materials for the fabrication of load-bearing prostheses.

It has also been found that metal prostheses are not completely inert in the body. Body fluids act upon the metals causing them to slowly corrode by an ionizing process that thereby releases metal ions into the body. Metal ion release from the prosthesis is also related to the rate of wear of load bearing surfaces because the passive oxide film, which is formed on the surface, is constantly removed. The repassivation process constantly releases metal ions during the ionizing process. Furthermore, the presence of third-body wear (cement or bone debris) accelerates this process and microfretted metal particles increase friction.

The excellent corrosion resistance of zirconium has been known for many years. Zirconium displays excellent corrosion resistance in many aqueous and non-aqueous media and for this reason has seen an increased use in the chemical process industry and in medical applications. A limitation to the wide application of zirconium in these areas is its relatively low resistance to abrasion and its tendency to gall. This relatively low resistance to abrasion and the tendency to gall is also demonstrated in zirconium alloys.

U.S. Pat. No. 2,987,352 to Watson first disclosed a method of producing zirconium bearings with a specific form of oxidized zirconium as a surface layer. The specific form of oxidized zirconium is a blue-black or blue oxidized zirconium. The method of Watson was refined by Haygarth (U.S. Pat. No. 4,671,824) resulting in improved abrasion resistance and better dimensional control of the oxidized product. U.S. Pat. No. 5,037,438 to Davidson first demonstrated the many advantages that are realized through the use of the specific form of oxidized zirconium on zirconium and zirconium alloy substrates in prosthetic devices. Davidson extended this work to include surfaces subject to nitridation in U.S. Pat. No. 5,180,394. Other U.S. patents of Davidson (U.S. Pat. Nos. 5,152,794; 5,370,694; 5,372,660; 5,496,359; and 5,549,667) demonstrate the use of this specific form of zirconium oxide or zirconium nitride in other prosthetic application. All of the aforementioned patents of Davidson are incorporated by reference as though fully disclosed herein. The advantages of these surfaces include increased strength, low friction and high wear resistance. U.S. Pat. Nos 5,037,438 and 5,180,394 to Davidson, respectively, disclose a method of producing zirconium alloy prostheses with an oxidized zirconium surface and a nitrided zirconium surface. The work of Watson and Davidson teach a specific form of oxidized or nitrided zirconium which possesses all of the advantages of ceramic materials while maintaining the strength of metallic surfaces. While the present invention is not intended to be limited by theory, the oxide or nitride layer are believed to be characterized by the presence of free oxygen or nitrogen which diffuses into the interior of the material, near the metallic substrate. The resulting “diffusion hardened” surfaces have oxide or nitride layers that possess properties that combine the unique advantages of both ceramic and metal surface, while simultaneously minimizing the disadvantages of these materials. All of the U.S. patents cited above to Davidson, Watson, and Haygarth are incorporated by reference as though fully set forth herein. While the early work of Davidson focused on pure zirconium and alloys of zirconium in which zirconium was the predominant metal, later work has shown that this is not necessary in order to form the desired diffusion hardened oxide. For instance, an alloy of 74 wt % titanium, 13 wt % niobium and 13 wt % zirconium (“Ti-13-13”) will form the diffusion hardened oxidation layer used herein. Ti-13-13 is taught in U.S. Pat. No. 5,169,597 to Davidson et al. By effectively taking advantage of the unique properties of such diffusion-hardened layers on prosthetic devices, the useful service life of the device is greatly improved. The improvement was realized by improving the wear resistance of the contacting surfaces of an implant (most notably the articulating surfaces), thereby lengthening the useful service life of the implant.

Apart from the issue of wear, another important performance criterion for medical implants as it relates to service life is the degree of fixation stability. The integrity of the fixation stability of the implant in the implanted tissue is another major factor in the service life of the implant. Fixation stability is typically accomplished through ingrowth of surrounding tissue into the implant and its ability to become firmly anchored to other components such as bone cement with a large shear strength. A typical hip joint prosthesis includes a stem fixated into the femur, a femoral head, and an acetabular cup against which the femoral head articulates. A typical knee joint prosthesis has a femoral and tibial component, both of which are fixated to their respective bones. This fixation could be to any tissue, and it is oftentimes assisted through the use of other materials, such as bone cement, etc.

Because of the improvements in wear resistance realized through the use of diffusion-hardened surfaces, fixation stability remains as a major limiting factor, among others, in the overall service life of implants. Fixation stability of the oxidized and nitrided zirconium prostheses of Davidson was accomplished through the use of porous metal beads or wire mesh coatings that promoted bone in-growth. These methods relied exclusively on an increase in the surface area for improving bone in-growth and on-growth into the implant. These techniques are taught in U.S. Pat. Nos. 5,037,438 and 5,180,394 as well as other patents of Davidson, and when combined with the advantages of oxidized or nitrided zirconium, represented an improvement in performance of medical implants in numerous areas. Nevertheless, these fixation methods have not kept up with the breakthrough advancement in prosthesis service life realized through the use of diffusion-hardened oxide surfaces such as oxidized zirconium. Fixation stability remains a weak link in the chain in the goal of long service life prosthetic devices. Accordingly, continued improvement in the fixation stability of such implants is desirable.

Recent efforts at improving fixation stability have been directed toward the use of textured surfaces. These techniques typically involve the use of chemical or electrochemical etching. Examples in the prior art include the U.S. patents of Wagner, (U.S. Pat. Nos. 5,922,029; 5,258,098; 5,507,815: and 6,193,762) in which the etching methodology is used. Although the techniques of Wagner et al. represent one potential source of methods for surface texture modification it is expected that any other surface texture modification techniques would be similarly useful in aiding fixation. For example, mechanical etching would also produce an acceptable textured surface. Notably, in copending U.S. Application No. 60/338,420, the use of textured surfaces is combined with diffusion-hardened oxidation surfaces to produce a prosthetic device having superior articulating surfaces and improved fixation stability.

Bioceramics in general, calcium phosphates, and hydroxyapatite in particular, have been used to promote bone growth. These chemical species are similar in chemical composition to bone and teeth. Much attention has been given in the art to the development of apatite materials to assist in the regeneration of bone defects and injuries. Very early on, similarities had been observed between the powder X-ray diffraction pattern of the in vivo mineral and hydroxyapatite. Calcium compounds, or calcium slats, including calcium sulfate (Nielson, 1944), calcium hydroxide (Peltier, 1957), and tricalcium phosphate (Albee et al., 1920), have been observed to stimulate new bone growth when implanted or injected into bone cavities (Hulbert et al., 1983). These materials also exhibit good biocompatibility and compositional similarities to human bone and tooth and can serve as resorbable or non-resorbable implants depending on their degree of microporosity. They have also been used as coatings on conventional implant devices (See e.g., U.S. Pat. Nos. 6,350,126; 6,261,322; 5,279,831; 5,164,187; and 5,039,546). The chemical similarity between the calcium-based bioceramics and the material found in natural bone is believed to be the mechanism by which these chemical species promote bone growth.

While all of these advancements have aided in the principle goal of lengthening of the useful life of prosthetic devices, further improvements are needed. A delay or complete prevention of failure of an implant avoids the need for revision surgery and is always desirable. There exists a need for a method to produce medical implants combining good fixation and low wear. This invention herein relates to metallic orthopedic implants having surfaces of a thin, dense, highly wear-resistant coating of diffusion-hardened oxidation or nitridation layer in addition to surfaces coated with one or more bioceramic or bone growth promoting materials such as one or more apatite compounds. The oxidation layer is formed by an in-situ process characterized by the diffusion of oxygen or nitrogen into the surface toward the unoxidized substrate below. The combination of high-strength, highly wear-resistant diffusion-hardened prosthetic surfaces with bioceramic-coated surfaces produces a prosthetic device with exceptionally long service life. The combination synergistically improves the implants' service life by addressing the two major failure mechanisms: wear of the articulating surfaces and implant loosening.

### SUMMARY OF THE INVENTION

In one aspect of the present invention, a prosthesis comprises a femoral component having an implant portion for inserting into body tissue and a bearing surface comprising at least one condyle. The femoral component is formed of zirconium, hafnium, niobium, tantalum or alloys of any of those metals. The prosthesis also comprises a tibial component having an articulating surface, the articulating surface being comprised of an organic polymer or polymer-based composite and is adapted to cooperate with said bearing surface. The prosthesis also has a diffusion-hardened oxide or nitride coating on at least a part of said bearing surface for reducing wear of the tibial component and has at least one bioceramic compound coating at least a part of said implant portion.

In a specific embodiment, the diffusion-hardened oxide or nitride coating is selected from the group consisting of oxidized zirconium, oxidized hafnium, oxidized niobium, oxidized tantalum, nitrided zirconium, nitrided hafnium, nitrided niobium, nitrided tantalum and combinations thereof.

In a preferred embodiment, the femoral component is formed of zirconium or zirconium alloy and the diffusion-hardened oxide coating comprises blue-black or black oxidized zirconium.

In an alternative embodiment, the tibial component further comprises an attachment portion formed of zirconium, hafnium, niobium, tantalum, or alloys thereof. The attachment portion may have a diffusion-hardened oxide or nitride coating, and that coating may be oxidized zirconium, oxidized hafnium, oxidized niobium, oxidized tantalum, nitrided zirconium, nitrided hafnium, nitrided niobium, nitrided tantalum or combinations thereof.

In a preferred embodiment, the attachment portion is comprised of zirconium or zirconium alloy and the diffusion-hardened oxide coating is made of blue-black or black oxidized zirconium.

In other embodiments, the bioceramic compound may be one or more of the compounds hydroxyapatite, fluoroapatite, chloroapatite, bromoapatite, iodoapatite, calcium sulfate, calcium phosphate, calcium carbonate, calcium tartarate, bioactive glass, and combinations thereof. In a preferred embodiment, the compound comprises hydroxyapatite.

In another embodiment, a prosthesis comprises a femoral component having an implant portion for inserting into body tissue, a head portion comprising a bearing surface, the femoral component being formed of zirconium, hafnium, niobium, tantalum or alloys thereof. The prosthesis also has an acetabular cup having an inner surface comprising an organic polymer or a polymer-based composite and an outer surface, the inner surface being adapted to cooperate with said bearing surface The prosthesis also has a diffusion-hardened oxide or nitride coating on at least a part of said bearing surface for reducing wear of said inner surface and at least one bioceramic compound coating on at least a part of the implant portion or the outer surface or both the implant portion and said outer surface.

In a specific embodiment, the prosthesis has a diffusion-hardened oxide or nitride coating which is selected from the group consisting of oxidized zirconium, oxidized haffium, oxidized niobium, oxidized tantalum, nitrided zirconium, nitrided hafnium, nitrided niobium, nitrided tantalum, and combinations thereof.

In a preferred embodiment, the femoral component is formed of zirconium or zirconium alloy and the diffusion-hardened oxide coating is made up of blue-black or black oxidized zirconium.

In an alternative embodiment, the outer surface formed of zirconium, hafnium, niobium, tantalum or alloys thereof. The outer surface of the device may be at least partly comprised of a diffusion-hardened oxide or nitride coating which may be oxidized zirconium, oxidized hafnium, oxidized niobium, oxidized tantalum, nitrided zirconium, nitrided hafnium, nitrided niobium, nitrided tantalum, or combinations thereof.

In a preferred embodiment, the outer surface is comprised of zirconium or zirconium alloy. In a specific embodiment, the diffusion-hardened oxide coating is made up of blue-black or black oxidized zirconium.

In other embodiments, the bioceramic compound may be one or more of hydroxyapatite, fluoroapatite, chloroapatite, bromoapatite, iodoapatite, calcium sulfate, calcium phosphate, calcium carbonate, calcium tartarate, bioactive glass, and combinations thereof. In a preferred embodiment, the compound comprises hydroxyapatite.

In a general embodiment of the invention, a prosthesis comprises a body having an implant portion for inserting into body tissue, the body being formed of zirconium, hafnium, niobium, tantalum or alloys thereof. The prosthesis also has a bearing surface on said body, the bearing surface being sized and shaped to engage or cooperate with a second bearing surface, the second bearing surface being a part of another prosthesis portion. The prosthesis also has a diffusion-hardened oxide or nitride coating on said bearing surface of said body and at least one bioceramic compound coating at least a part of said body.

In a specific embodiment, the diffusion-hardened oxide coating is selected from the group consisting of oxidized zirconium, oxidized hafnium, oxidized niobium, oxidized tantalum, nitrided zirconium, nitrided hafnium, nitrided niobium, nitrided tantalum, and combinations thereof.

In a preferred embodiment, the body is formed of zirconium or zirconium alloy and the diffusion-hardened oxide or nitride coating comprises blue-black or black oxidized zirconium.

In an alternative embodiment, the other prosthesis portion comprises zirconium, hafnium, niobium, tantalum, or alloys thereof. In a specific embodiment, the other prosthesis portion comprises a diffusion-hardened oxide or nitride coating. In yet another specific embodiment, the diffusion-hardened oxide or nitride coating of the other prosthesis portion comprises oxidized zirconium, oxidized hafnium, oxidized niobium, oxidized tantalum, nitrided zirconium, nitrided hafnium, nitrided niobium, nitrided tantalum, or combinations thereof.

In a preferred embodiment, the other prosthesis portion comprises zirconium or zirconium alloy. It may have a diffusion-hardened oxide coating made up of blue-black or black oxidized zirconium.

In a specific embodiment, the bioceramic compound on the body may be one or more of hydroxyapatite, fluoroapatite, chloroapatite, bromoapatite, iodoapatite, calcium sulfate, calcium phosphate, calcium tartarate, bioactive glass, and combinations thereof. In a preferred embodiment, the compound comprises hydroxyapatite.

In an alternative embodiment, the other prosthesis portion comprises a coating of at least one bioceramic compound.

In a specific embodiment, the coating of at least one bioceramic compound on the other prosthesis portion comprises a compound selected from the group consisting of hydroxyapatite, fluoroapatite, chloroapatite, bromoapatite, iodoapatite, calcium sulfate, calcium phosphate, calcium tartarate, bioactive glass, and combinations thereof. Preferably, the compound comprises hydroxyapatite.

In a preferred embodiment, the second bearing surface comprises an organic polymer or polymer composite.

In another embodiment, a prosthesis comprises a body having an implant portion for inserting into the body tissue of a patient, the body being formed of zirconium, hafnium, niobium, tantalum or alloys thereof. The prosthesis also has a bearing surface on the body, a counter bearing surface adapted to cooperate with the bearing surface, a diffusion-hardened oxide or nitride coating at least a part of said bearing surface, and at least one bioceramic compound coating at least a part of said implant portion.

In a specific embodiment, the diffusion-hardened oxide or nitride coating is selected from the group consisting of oxidized zirconium, oxidized niobium, oxidized hafnium, oxidized tantalum, nitrided zirconium, nitrided hafnium, nitrided niobium, nitrided tantalum, and combinations thereof.

In a preferred embodiment, the body is formed of zirconium or zirconium alloy and the diffusion-hardened oxide coating comprises blue-black or black oxidized zirconium.

In an alternative embodiment, the counter bearing surface is comprised of a diffusion-hardened oxide or nitride coating. The diffusion-hardened oxide or nitride coating on the counter bearing surface may be selected from the group consisting of oxidized zirconium, oxidized niobium, oxidized hafnium, oxidized tantalum, nitrided zirconium, nitrided hafnium, nitrided niobium, nitrided tantalum, and combinations thereof.

In a preferred embodiment, the counter bearing comprises blue-black or black oxidized zirconium.

In one embodiment, the at least one bioceramic compound comprises a compound selected from the group consisting of hydroxyapatite, fluoroapatite, chloroapatite, bromoapatite, iodoapatite, calcium sulfate, calcium phosphate, calcium tartarate, bioactive glass, and combinations thereof. Preferably, the compound comprises hydroxyapatite.

In a preferred embodiment, the counter bearing surface comprises an organic polymer or polymer composite.

In another embodiment, the prosthesis further comprises an irregular surface formed of beads of zirconium, hafnium, niobium, tantalum, or alloys thereof. In a specific embodiment, the prosthesis further comprises a diffusion-hardened surface on the beads or a coating of at least one apatite compound on the beads or both a diffusion-hardened surface and a coating of at least one bioceramic compound on the beads.

In another embodiment, the prosthesis further comprises an irregular surface formed of wire mesh of zirconium, hafnium, niobium, tantalum, or alloys thereof. In a specific embodiment, the prosthesis further comprises a diffusion-hardened surface on the wire mesh or a coating of at least one bioceramic compound on the wire mesh or both a diffusion-hardened surface and a coating of at least one bioceramic compound on the wire mesh.

In another embodiment, the prosthesis further comprises a textured surface formed of zirconium, hafnium, niobium, tantalum, or alloys thereof. In a specific embodiment, the prosthesis further comprises a diffusion-hardened surface on said textured surface or a coating of at least one bioceramic compound on said textured surface or both a diffusion-hardened surface and a coating of at least one bioceramic compound on said textured surface.

In another embodiment of the present invention, an endoprosthesis is described. In this embodiment, the prosthesis comprises a prosthesis body formed of zirconium, hafnium, niobium, tantalum or alloys thereof, and the prosthesis body forming one component of a two-component joint and having a bearing surface at least a portion of which is adapted to cooperate with and slide against body tissue of a second joint component. The prosthesis also comprises a diffusion-hardened oxide or nitride coating on at least a part of a bearing surface adapted to cooperate and slide against the body tissue, said coating selected from the group consisting of oxidized zirconium, oxidized hafnium, oxidized niobium, oxidized tantalum, nitrided zirconium, nitrided hafnium, nitrided niobium, nitrided tantalum, and combinations thereof. The prosthesis of this embodiment also comprises at least one bioceramic compound coating on at least a part of said prosthesis body.

In a specific embodiment, the bearing surface is a femoral head adapted to cooperate with and slide against cartilage tissue of a pelvis.

In another embodiment, the bearing surface is a head of a humeral implant adapted to cooperate with natural body tissue of a glenoid of a recipient.

In an alternative embodiment, the bearing surface is a bearing surface of a glenoid prosthesis adapted to cooperate with natural tissue of a humerus.

In another embodiment, the bearing surface is a bearing surface of at least one condyle of a femoral component of a knee joint prosthesis adapted to cooperate against natural tissue of a tibia.

In an alternative embodiment, the bearing surface is a bearing surface of a tibial component of a knee joint prosthesis adapted to cooperate with natural tissue of condyles.

Various other embodiments include those wherein the at least one bioceramic compound is selected from the group consisting of hydroxyapatite, fluoroapatite, chloroapatite, bromoapatite, iodoapatite, calcium sulfate, calcium phosphate, calcium tartarate, bioactive glass, and combinations thereof.

In a preferred embodiment, the prosthesis body is formed of zirconium or alloys thereof and the diffusion-hardened oxide coating comprises blue-black or black oxidized zirconium.

In another embodiment of the present invention, there is a prosthesis comprising a body formed of alloy having a composition comprising from about 10 to about 20 wt % niobium or from about 35 to about 50 wt % niobium; from about 13 to about 20 wt % zirconium; and the balance titanium; a diffusion-hardened oxide or nitride coating on at least a part of the body; and at least one bioceramic compound coating at least a part of the body.

In a specific embodiment, the prosthesis has a composition consisting essentially of about 74 wt % titanium, about 13 wt % niobium, and about 13 wt % zirconium.

### DESCRIPTION OF THE DRAWINGS

FIG. 1. Typical hip prosthesis shown in vivo.

FIG. 2. Typical hip prosthesis shown ex vivo.

FIG. 3. Typical knee prosthesis shown in vivo.

FIG. 4. Typical knee prosthesis shown ex vivo.

### DETAILED DESCRIPTION OF THE INVENTION

As used herein, “a” or “an” may mean one or more. As used herein in the claim(s), when used in conjunction with the word “comprising”, the words “a” or “an” may mean one or more than one. As used herein, “another” may mean at least a second or more.

As used herein, “apatite” means any chemical species of the genus having the empirical formula Ca₅(PO₄)₃X where X is any univalent ligand satisfying the electroneutrality of the general formula. “Hydroxyapatite” is defined as the apatite wherein X=OH, or having the empirical formula Ca₅(PO₄)₃OH.

As used herein, “bioceramic” means any ceramic material including apatites (hydroxyapatite, fluoroapatite, chloroapatite, bromoapatite, and iodoapatite), calcium sulfate, calcium phosphate, calcium carbonate, calcium tartarate, bioactive glass, and combinations thereof.

As used herein, the term, bone growth-promoting material means any material that promotes growth of bone tissue by any mechanism. These include, but are not limited to, the bioceramic materials defined above.

As used herein, as it refers to interacting surfaces on a prosthetic device, the term “cooperate” is defined as any type of interaction, including an articulating interaction, a non-articulating interaction, and any and all intermediate levels of interaction.

As used herein, “diffusion-hardened surface” is defined as a type of abrasion resistant surface formed by certain specific in-situ oxidation or nitridation processes. The surface is characterized by being oxidized or nitrided relative to the substrate upon which it is situated. It is oxidized or nitrided by an in-situ oxidation or nitridation process by which oxygen or nitrogen diffuses from the surface toward the interior substrate domain. Specific examples of the oxidation or nitridation processes are provided herein. When used in reference to the underlying substrate material, it is synonymous with “surface hardened”. Also synonymously, the surface oxide or nitride layer is also referred to as “diffusion-bonded”. An oxidized or nitrided zirconium surface, as those terms are used herein, are examples of a diffusion hardened surface; other metals or metal alloys may also form diffusion-hardened surfaces by oxidation or nitridation. In all discussions herein referring to various applications and embodiments of diffusion-hardened surfaces on prosthetic devices, it should be understood that discussions with respect to oxidized surfaces apply equally to nitrided surfaces.

As used herein, “metallic” may be a pure metal or an alloy.

As used herein, “nitridation” is defined as the chemical process by which a substrate material, preferably a metal is combined with nitrogen to form the corresponding nitride.

As used herein, “zirconium alloy” is defined as any metal alloy containing zirconium in any amount greater than about 10% by weight of zirconium. Thus, an alloy in which zirconium is a minor constituent at about 10% by weight or greater is considered a “zirconium alloy” herein. Similarly, a “metal alloy” of any other named metal (e.g., a hafnium alloy or a niobium alloy; in these cases, the named metal is hafnium and niobium, respectively) is defined as any alloy containing the named metal in any amount greater than about 10% by weight.

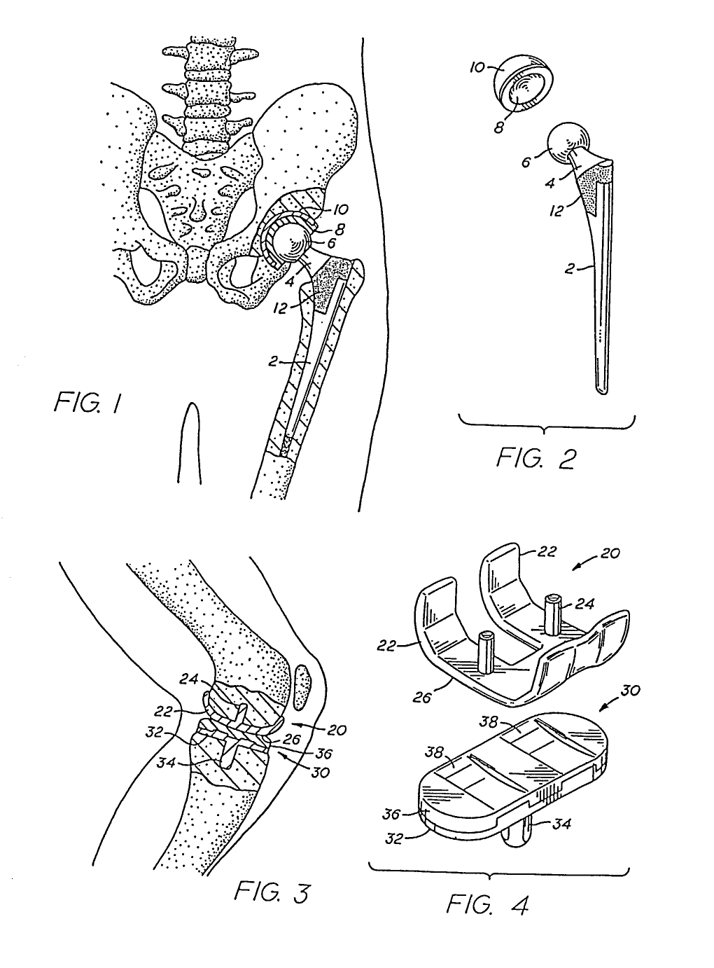

One aspect of the invention is to combine low friction, wear resistant surfaces with surfaces which promote bone in-growth and on-growth. Illustrative examples of such articulating surfaces are shown in the schematic diagrams, FIGS. 1-4.

A typical hip joint assembly is shown in situ in FIGS. 1 and 2. The hip joint stem 2 fits into the femur while the femoral head 6 of the prosthesis fits into and articulates against the inner lining 8 of an acetabular cup 10 which in turn is affixed to the pelvis as shown in FIG. 1. In prior art devices, a porous metal bead or wire mesh coating 12 is incorporated to allow stabilization of the implant by ingrowth of surrounding tissue into the porous coating. More recently, textured surfaces have been employed in various surfaces which directly contact bone (such as area 12), in order to increase surface area and allow the implant to dig in to the bone. Similarly, such a coating can also be applied to the outer surface (contacting the pelvis) of the acetabular component. The femoral head 6 may be an integral part of the hip joint stem 2 or may be a separate component mounted upon a conical taper at the end of the neck 4 of the hip joint prosthesis. This allows the fabrication of a prosthesis having a metallic stem and neck but a femoral head of some other material, such as ceramic. This method of construction is often desirable because ceramics have been found to generate less frictional torque and wear when articulating against the lining of an acetabular cup. The lining is typically formed of ultra-high molecular weight polyethylene (UHMWPE) or cross-linked polyethylene (XLPE); however, other suitable materials may be used. Regardless of the materials, however, the femoral head articulates against the inner surface of the acetabular cup thereby causing wear and, in the long term, this may necessitate prosthesis replacement. This is especially the case where the femoral head is of metal and the acetabular cup is lined with an organic polymer or composite thereof. While these polymeric surfaces provide good, relatively low friction surfaces and are biocompatible, they are subject to wear and accelerated creep due to the frictional heat and torque to which they are subjected during ordinary use. The use of a diffusion-hardened oxide layer surface such as oxidized zirconium significantly extends the useful service life of the articulating couple.

A typical knee joint prosthesis is shown in situ in FIGS. 3 and 4. The knee joint includes a femoral component 20 and a tibial component 30. The femoral component includes condyles 22 which provide the articulating surface of the femoral component and pegs 24 for affixing the femoral component to the femur. The pegs (24) and the surfaces adjacent to the pegs directly contact the femur. The pegs (24) and the adjacent surfaces have been subjected to the same stabilization techniques as were discussed for hip prostheses; i.e., porous metal beads, wire mesh coatings, and more recently, textured surfaces. The tibial component 30 includes a tibial base 32 with a peg 34 for mounting the tibial base onto the tibia. A tibial platform 36 is mounted atop the tibial base 32 and is supplied with grooves 38 similar to the shape of the condyles 22. The bottom surfaces of the condyles 26 contact the tibial platform's grooves 38 so that the condyles articulate within these grooves against the tibial platform. While condyles are typically fabricated of metals, the tibial platform may be made from an organic polymer or a polymer-based composite. Thus, the hard metallic condyle surfaces 26 would articulate against a relatively softer organic composition. As previously explained, this may result in wear of the organic material, i.e. the tibial platform necessitating the replacement of the prosthesis. As in the case of the hip joint, porous bead or wire mesh coatings can also be applied to either the tibial or femoral components of the knee or both.

The invention provides orthopedic implants having diffusion-hardened oxide or nitride surfaces such as oxidized zirconium or nitrided zirconium. More generally, metals or metal alloys of titanium, vanadium, niobium, hafnium and/or tantalum may be used as substrate materials to form suitable diffusion-hardened oxide surface layers. Most of the examples herein deal with zirconium or zirconium alloy substrates and surface layers of oxidized zirconium or nitrided zirconium; however, it should be understood that other metals such as hafnium, vanadium, titanium, niobium, tantalum, and their alloys, are amenable to the present invention. In order to form continuous and useful oxide or nitride coatings over the desired surface of the metal alloy prosthesis substrate, the metal alloy should preferably contain from about 80 to about 100 wt. % of the desired metal, and more preferably from about 95 to about 100 wt. %. It should be noted that in some cases, lower amount of the desired metal are possible. In some cases, alloys where the desired metal is at about 10% by weight or greater may yield acceptable results. For example, an alloy of about 74 wt % titanium, about 13 wt % niobium and about 13 wt % zirconium (“Ti-13-13”) can be successfully used herein. Ti-13-13 is taught in U.S. Pat. No. 5,169,597 to Davidson et al. Thus, while levels of the desired metal of about 10% by weight or greater are known to produce acceptable results, increasing this level continuously gives progressively better results, with at least 80% by weight, and at least 95% by weight, being the preferred and most preferred levels, respectively.

In the case of either oxidized or nitrided zirconium, oxygen, niobium, and titanium, among others, may be included as common alloying elements in the alloy with often times the presence of hafnium. Yttrium may also be alloyed with the zirconium to enhance the formation of a tougher, yttria-stabilized zirconium oxide coating during the oxidation of the alloy. While oxidized or nitrided zirconium is used for illustrative purposes herein, it should be understood that the teachings apply analogously to the other possible metal candidates as well. While such zirconium containing alloys may be custom formulated by conventional methods known in the art of metallurgy, a number of suitable alloys are commercially available. In the case of oxidized zirconium. some commercial alloys include, among others Zircadyne 705, Zircadyne 702, and Zircalloy.

The base metal and metal alloys are cast or machined by conventional methods to the shape and size desired to obtain a suitable prosthesis substrate. The substrate is then subjected to process conditions which cause the in situ formation of a tightly adhered, diffusion-bonded coating of zirconium oxide or zirconium nitride on its surface. The term “diffusion-hardened” and “diffusion-bonded” are used in reference to the desired oxides or nitrides because the formation of these particular surfaces is characterized by the diffusion of oxygen or nitrogen from the surface towards the interior (i.e., approaching the unoxidized substrate, native metal or metal alloy). It is believed that this diffusion of oxygen or nitrogen is what imparts the high strength and high wear resistance to these surfaces. The process conditions for formation include, for instance, air, steam, or water oxidation or oxidation in a salt bath. These processes ideally provide a thin, hard, dense, low friction, wear-resistant zirconium nitride or blue-black or black wear-resistant zirconium oxide film or coating of thicknesses typically on the order of several microns (10⁻⁶ meter) on the surface of the prosthesis substrate. Below this coating, diffused oxygen or nitrogen from the oxidation or nitridation process increases the hardness and strength of the underlying substrate metal.

The air, steam and water oxidation processes are described for zirconium and zirconium alloys in now-expired U.S. Pat. No. 2,987,352 to Watson, the teachings of which are incorporated by reference as though fully set forth. These methods may also be applied to metals and alloys of hafnium, titanium, vanadium, niobium, and tantalum. In the case of zirconium or zirconium alloy, the air oxidation process provides a firmly adherent black or blue-black layer of zirconium oxide of highly oriented monoclinic crystalline form. If the oxidation process is continued to excess, the coating will whiten and separate from the metal substrate. The oxidation step may be conducted in either air, steam or hot water. For convenience, the metal prosthesis substrate may be placed in a furnace having an oxygen-containing atmosphere (such as air) and typically heated at 700° F.-1100° F. up to about 6 hours. However, other combinations of temperature and time are possible. When higher temperatures are employed, the oxidation time should be reduced to avoid the formation of the white oxide.

The oxide layer should range in thickness from about 1 to about 20 microns; however, a range of from about 1 to about 5 microns is preferred. The overall average thickness can be controlled by the parameters of time and temperature. For example, furnace air oxidation at 1000° F. for 3 hours will form an oxide coating on Zircadyne 705 about 2-3 microns thick, oxidation at 1175° F. for 1 hour results in an overall average oxide coating of about 4-5 microns thick, and oxidation at 1175° F. for 3 hours results in an overall average oxide coating of about 10-11 microns thick. As additional examples, one hour at 1300° F. will form an oxide coating about 14 microns in thickness, while 21 hours at 1000° F. will form an oxide coating thickness of about 9 microns. Using different combinations of oxidation times and higher oxidation temperatures will increase or decrease this thickness, but higher temperatures and longer oxidation times may compromise coating integrity, depending upon the nature of the substrate and other factors. For thicker coatings of oxide, some trial and error may be necessary. Of course, because in the usual case only a thin oxide is necessary on the surface, only very small dimensional changes, typically less than 10 microns over the thickness of the prosthesis, will result. In general, thinner coatings (1-4 microns) have better attachment strength.

One of the salt-bath methods that may be used to apply the oxide coatings to the metal alloy prosthesis, is the method of U.S. Pat. No. 4,671,824 to Haygarth, the teachings of which are incorporated by reference as though fully set forth. In the case of oxidized zirconium, the salt-bath method provides a similar, slightly more abrasion resistant blue-black or black zirconium oxide coating. The method requires the presence of an oxidation compound capable of oxidizing zirconium in a molten salt bath. The molten salts include chlorides, nitrates, cyanides, and the like. The oxidation compound, sodium carbonate, is present in small quantities, up to about 5 wt %. The addition of sodium carbonate lowers the melting point of the salt. As in air oxidation, the rate of oxidation is proportional to the temperature of the molten salt bath and the '824 patent prefers the range 550° C.-800° C. (1022° F.-1470° F.). However, the lower oxygen levels in the bath produce thinner coatings than for furnace air oxidation at the same time and temperature. A salt bath treatment at 1290° F. for four hours produces an oxide coating thickness of roughly 7 microns.

Whether air oxidation in a furnace or salt bath oxidation is used, the oxide coatings are quite similar in hardness. For example, if the surface of a wrought Zircadyne 705 (Zr, 2-3 wt. % Nb) prosthesis substrate is oxidized, the hardness of the surface shows a dramatic increase over the 200 Knoop hardness of the original metal surface. The surface hardness of the resulting blue-black zirconium oxide surface following oxidation of Zircadyne 705 by either the salt bath or air oxidation process is approximately 1700-2000 Knoop hardness.

In the case of nitridation of zirconium and zirconium alloys, an analogous procedure is used. As in the oxide case, the nitride layer should range in thickness from about 1 to about 20 microns; however, a range of from about 1 to about 5 microns is preferred. Even though air contains about four times as much nitrogen as oxygen, when zirconium or a zirconium alloy is heated in air as described above, the oxide coating is formed in preference to the nitride coating. This is because the thermodynamic equilibrium favors oxidation over nitridation under these conditions. Thus, to form a nitride coating the equilibrium must be forced into favoring the nitride reaction. This is readily achieved by elimination of oxygen and using a nitrogen or ammonia atmosphere instead of air or oxygen when a gaseous environment (analogous to “air oxidation”) is used. In order to form a zirconium nitride coating of about 5 microns in thickness, the zirconium or zirconium alloy prosthesis should be heated to about 800° C. for about one hour in a nitrogen atmosphere. Thus, apart from the removal of oxygen (or the reduction in oxygen partial pressure), or increasing the temperature, conditions for forming the zirconium nitride coating do not differ significantly from those needed to form the blue-black or black zirconium oxide coating. Any needed adjustment would be readily apparent to one of ordinary skill in the art.

When a salt bath method is used to produce a nitride coating, then the oxygen-donor salts should be replaced with nitrogen-donor salts, such as, for instance cyanide salts. Upon such substitution, a nitride coating may be obtained under similar conditions to those needed for obtaining an oxide coating. Such modifications as are necessary, may be readily determined by those of ordinary skill in the art. Alternatively, the zirconium nitride may be deposited onto the zirconium or zirconium alloy surface via standard physical or chemical vapor deposition methods, including those using an ion-assisted deposition method. It is preferred that the physical or chemical vapor deposition methods be carried out in an oxygen-free environment. Techniques for producing such an environment are known in the art, for instance the bulk of the oxygen may be removed by evacuation of the chamber and the residual oxygen may be removed with an oxygen scavenger.

When the zirconium or zirconium alloy is provided with a zirconium porous bead, zirconium wire mesh surface, or textured surface, then this surface layer can also be coated with zirconium oxide or nitride, as the case may be, to provide protection against metal ionization in the body.

These diffusion-bonded, low friction, highly wear resistant oxidized or nitrided zirconium coatings are grown in-situ and used on the surfaces of orthopedic implants subject to conditions of wear. Such surfaces include, but are not limited to, the articulating surfaces of knee joints, elbows and hip joints. As mentioned before, in the case of hip joints, the femoral head and stem are typically fabricated of metal alloys while the acetabular cup may be fabricated from ceramics, metals or organic polymer-lined metals or ceramics. However, the acetabular cup may be fabricated of a metal or metal alloy that forms a diffusion-hardened surface.

When the diffusion-hardened oxide or nitride coated femoral head is used in conjunction with any of these acetabular cups, the coefficient of friction between the femoral head and the inner surface of the cup is reduced so that less heat and torque is generated and less wear of the mating bearing surface results. This reduction in heat generation, frictional torque, and wear is particularly important in the case of acetabular cups lined with organic polymers or composites of such polymers. Organic polymers, such as UHMWPE, exhibit rapidly increased rates of creep when subjected to heat with consequent deleterious effect on the life span of the liner. Wear debris of the polymer leads to adverse tissue response and loosening of the device. The diffusion-hardened coating serves to protect the prosthesis substrate and increase its mechanical strength properties but, as a result of its low friction surface, it also protects those surfaces against which it is in operable contact and consequently enhances the performance and life of the prosthesis.

The usefulness of prostheses employing diffusion-hardened surfaces is not limited to load-bearing surfaces of load-bearing prostheses, but are also applicable to non-load bearing prostheses, especially joints, where a high rate of wear may be encountered. Because the diffusion-hardened surface is firmly bonded to the metal or metal alloy prosthesis substrate, it provides a barrier between the body fluids and the metal or metal alloy, thereby preventing the corrosion of the alloy by the process of ionization and its associated metal ion release. Because these diffusion-hardened surfaces offer advantages in both mechanical wear and for the prevention of metal ion release, they are applicable to any and all surfaces of a prosthetic device.

The substrate metal or metal alloy has been used to provide a porous bead, wire mesh, or textured surface to which surrounding bone or other tissue may integrate to stabilize the prosthesis. The porous metal beads, wire mesh or textured surface can have diffusion-hardened surfaces as well. As a result, these special surfaces can be rendered non-ion releasing in a way similar to the oxidation or nitridation of the base prosthesis for the elimination or reduction of metal ion release. These roughened surfaces improve bone ingrowth and on-growth by providing an increased surface area for adhesion, and by providing an increased surface area onto which bone in-growth and on-growth may occur.

The inventors have discovered that extending the useful service life of prosthetic devices can be realized by combining the advantages of diffusion-hardened surfaces with the fixation stability imparted by the use of bioceramic compounds. These material surfaces enhance fixation by both a physical/mechanical mechanism (increasing surface area for better adhesion) and a chemical mechanism (e.g., the promotion of bone growth by apatites in general and hydroxyapatites in particular). The porous metal beads/wire mesh/textured surface techniques operate only by the physical/mechanical mechanism of increasing surface area for better adhesion. In this way, bioceramics have better fixation-enhancing abilities by virtue of their multiple modes of action. In the case of apatite compounds, the apatite surfaces interact with bone or provide for bone ingrowth enhancing the fixation stability of the device. Because of the chemical similarities between the apatites and the natural material in bones, it is believed that there is a chemical driving force which promotes bone growth in the presence of apatites. Coating with apatite can also increase the attachment area on the implant which is available for bone in-growth and on-growth area while simultaneously chemically promoting bone growth. This dual mechanism of promoting bone in-growth and on-growth results in improved fixation stability of implants employing apatite coatings. Bioceramics in general exhibit similar beneficial properties. The inventors have applied these synergies for the fabrication of exceptionally long-life prosthetic devices.

The biocermaic or apatite coatings may be produced by any conventional or non-conventional means. One of ordinary skill in the art is familiar with these methods, particularly those employing apatite compounds, especially hydroxyapatite. For the purposes of discussion, much of the remainder of the discussion focuses on the apatite compounds with the understanding that the invention is not so limited. Use of other bioceramic and bone growth-promoting materials is largely analogous, with only minor modifications perhaps necessary and otherwise known to one of ordinary skill in the art. These other materials include, but are not limited to, calcium sulfate, calcium phosphate, calcium carbonate, calcium tartarate, bioactive glass, and combinations thereof.

In the preferred embodiment, an apatite coating is used and the apatite coatings will be hydroxyapatite, Ca₅(PO₄)₃(OH), and possess a large surface area owing to the fibrous nature of the hydroxyapatite crystals. The surface area will generally range from about 1-25 m²/cm² of area. The coatings may be as thin as about 2 μm, preferably being at least about 5 μm, and more preferably at least about 10 μm, and may range to 40 μm thick or greater, depending upon need. Usually, a relatively thin coating will be employed to avoid thick brittle ceramic interfaces between the substrate and the ductile bone. The high surface of this coating presents orders of magnitude more binding surface than the uncoated implant or the conventional calcium phosphate coatings.

The apatite composition may be modified in a variety of ways by the introduction of other ions, as required. Other ions include fluoride, carbonate, sodium, chloride, hydrogen ions, HPO₄, HCO₃, etc., and the like. Usually fewer than about 50%, more usually fewer than about 20% of the total number of phosphate and hydroxide anions and up to 50% of calcium cation will be substituted with other ions. These substitutions will influence the in vivo dissolution behavior of the coating which may be resorbable or non-resorbable.

Hydroxyapatite possesses a net positive charge at physiological pH which attracts negatively charged proteins, such as collagen or other exogenous or endogenous proteins, which may serve as growth factors and result in other interfacial chemistry. Thus, the coating may provide for the presence of such products on the surface of the hydroxyapatite or analogs or as part of the structure of the hydroxyapatite.

The coatings may be applied by any conventional or non-conventional methods of applying bioceramic, and n particular, hydoxyapatite or apatite compounds. All patent references describing such techniques are incorporated by reference as though fully described herein. For example, the bioceramic may be applied to solid surfaces, porous surfaces, etched surfaces, or any other type of surface. Because the coating may be applied in a liquid medium which is able to penetrate channels, pores, indentations and other structural features, a uniform coating may be obtained which can coat substantially the entire surface, without leaving exposed areas. In one solution-based deposition of hydroxyapatite, small, sticky hydroxyapatite colloidal particles in suspension are formed in proximity to the substrate to be coated by the addition of calcium and phosphate reactants in solution. (See U.S. Pat. Nos. 5,188,670; 5,279,831; and 5,164,187). Alternatively, the bioceramic may be applied as dry particulates as taught in U.S. Pat. No. 4,693,986, which is incorporated by reference herein as though fully described. Vapor deposition techniques, plasma spray deposition (see U.S. Pat. No. 6,280,789), electrodeposition (U.S. Pat. No. 5,759,376) are additional illustrative examples of known methods for applying surface coatings of bioceramic compounds. The precise mode of deposition is unimportant and any and all means yielding a coating of apatite having good structural integrity are acceptable. In the preferred embodiment, the bioceramic compound or compounds are deposited via plasma spray deposition or chemical vapor deposition.

In the hip joint assembly shown in situ in FIGS. 1 and 2, areas 12 and 2 are, among others, examples of areas wherein the apatite coating of the present invention could be applied. In many conventional prosthetic devices, these areas, particularly area 12, comprise porous metal beads, wire mesh, and/or textured surfaces to enhance fixation stability. As per the present invention, these areas as well as other areas, could have an bioceramic coating (most preferably, a hydroxyapatite coating) in lieu of, or alternatively in addition to, the conventional porous metal beads, wire mesh coatings, and/or textured surfaces used to promote bone in-growth or on-growth. Positioning the coating on the femoral stem affords a good deal of surface area contact with the femor and allows stabilization of the implant by ingrowth of surrounding tissue into the porous coating. Similarly, such a coating can also be applied to the outer surface of the acetabular component. The femoral head 6 may be an integral part of the hip joint stem 2 or may be a separate component mounted upon a conical taper at the end of the neck 4 of the hip joint prosthesis. This allows the fabrication of a prosthesis having a metallic stem and neck but a femoral head of some other material, such as ceramic. Regardless of the materials, however, the use of bioceramic coatings in any area where the prosthetic device contacts bone will result in enhanced fixation stability and allow prosthetic devices employing diffusion-hardened surfaces to realize the full service-enhancing attributes of those surfaces. In this way, fixation stability is no longer a major limiting factor in the effort to fabricate a truly life-long prosthetic devices employing diffusion-hardened oxide surfaces. The use of diffusion-hardened surfaces coupled with bioceramic-promoted fixations markedly extends the life of such prostheses.

As in the case of the hip joint, in a knee prosthesis, porous metal beads, wire mesh coatings, or textured surfaces can also be applied to either the tibial or femoral components of the knee or both. In the typical knee joint prosthesis shown in situ in FIG. 4, selected area within the femoral component 20, particularly those areas around the pegs 24 may be coated with bioceramics such as, for example, the haloapatites such as fluoroapatite and chloroapatite, but preferably hydroxyapatite. The tibial component 30 includes a tibial base 32 with a peg 24 for mounting the tibial base onto the tibia. The underside of the tibial base directly contacts the tibia and is an example of an ideal location for a coating of bioceramic. It should be noted that the aforementioned areas are non-limiting examples of areas wherein the bioceramic coating could be applied to a prosthetic having a diffusion-hardened oxidized or nitrided surface.

The invention described herein is also useful in endoprostheses or unipolar prostheses. These include, but are not limited to shoulder, knee, and hip endoprostheses. In these devices, the bearing surface cooperates with body tissue, most commonly cartilage. At least a part of the bearing surface of the endoprosthesis will have a diffusion-hardened oxide or nitride coating and at least part of the body of the endoprosthesis will have an coating of at least one bioceramic compound. The methods for applying the diffusion-hardened oxide or nitride coating are the same as those for other prosthetic devices. Similarly, the bioceramic coating may be applied in any manner, conventional or otherwise.

It is important to note that the areas where the bioceramics could be applied may vary, but it is preferred that the application occur in areas of maximum contact with bone, as such would promote maximum bone in-growth and on-growth. Bioceranic may or may not be applied on at least part of the diffusion-hardened surface, This would also be true in the case of other prosthetic devices such as shoulders, fingers, jaws, elbows, and others. The invention is broadly described to encompass any prosthetic device having at least part of its surface comprising a diffusion-hardened surface and at least part of its surface comprising one or more bioceramic materials.

The use of bioceramic coatings and diffusion-hardened surfaces on prosthetic devices can be performed in conjunction with conventional techniques for effecting fixation stability of such devices. These include, but are not limited to, the use of irregular surfaces of beads and/or wire mesh or the use of textured surfaces such as those known in the art and formed by techniques such as chemical, electrochemical, and/or mechanical etching. These conventional fixation surfaces may themselves comprise a diffusion-hardened surface or an bioceramic coating or both.

One skilled in the art readily appreciates that the present invention is well adapted to carry out the objectives and obtain the ends and advantages mentioned as well as those inherent therein. Systems, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the claims.

### U.S. Patent Documents

| | | |
|---|---|---|
| 2,987,352 | June 1961 | Watson |
| 4,671,824 | June 1987 | Haygarth |
| 4,673,409 | June 1987 | Van Kampen |
| 4,644,942 | February 1987 | Sump |
| 4,272,855 | June 1981 | Frey |
| 4,865,603 | September 1989 | Noiles |
| 5,922,029 | July 1999 | Wagner et al. |
| 5,507,815 | April 1996 | Wagner et al. |
| 5,258,098 | November 1993 | Wagner et al. |
| 6,193,762 | February 2001 | Wagner et al. |
| 5,037,438 | August 1991 | Davidson |
| 5,152,794 | October 1992 | Davidson |
| 5,169,597 | December 1992 | Davidson et al. |
| 5,180,394 | January 1993 | Davidson |
| 5,370,694 | December 1994 | Davidson |
| 5,372,660 | December 1994 | Davidson et al. |
| 5,496,359 | March 1996 | Davidson |
| 5,549,667 | August 1996 | Davidson |
| 5,188,670 | February 1993 | Constantz |
| 5,279,831 | January 1994 | Constantz |
| 5,164,187 | November 1992 | Constantz |

### Other References

Albee, et al., “Studies in Bone Growth,” Ann. Surg., 71:32-39, 1920.

Hulbert et al., “History of Bioceramics,” Ceramics in Surgery, 3-27, 1983.

Nielson, “Filling of Sterile and Infected Bone Cavities by Means of Plaster of Paris,” Acta Chir. Scandanav., 91:17-27, 1944.

Peltier, et al., “The Using of Plaster of Paris to Fill Defects in Bone,” Ann. Surg., 146:61-69, 1957.

What is claimed is:

1. A prosthesis comprising:
(a) a femoral component having
(1) an implant portion for inserting into body tissue;
(2) a bearing surface comprising at least one condyle;
said femoral component formed of zirconium, hafnium, niobium, tantalum or alloys thereof;
(b) a tibial component having an articulating surface, said articulating surface comprised of an organic polymer or polymer-based composite and adapted to cooperate with said bearing surface;
(c) a diffusion-hardened oxide or nitride coating on at least a part of said bearing surface for reducing wear of the tibial component; and,
(d) a coating of at least one bioceramic compound on at least a part of said implant portion.

2. The prosthesis of claim 1 wherein said diffusion-hardened oxide or nitride coating is selected from the group consisting of oxidized zirconium, oxidized hafnium, oxidized niobium, oxidized tantalum, nitrided zirconium, nitrided hafnium, nitrided niobium, nitrided tantalum and combinations thereof.

3. The prosthesis of claim 1 wherein said femoral component is formed of zirconium or zirconium alloy and said diffusion-hardened oxide or nitride coating comprises blue-black or black oxidized zirconium.

4. The prosthesis of claim 1 wherein said tibial component further comprises an attachment portion formed of zirconium, hafnium, niobium, tantalum, or alloys thereof.

5. The prosthesis of claim 4 wherein at least a part of said attachment portion is comprised of a diffusion-hardened oxide or nitride coating.

6. The prosthesis of claim 5 wherein said diffusion-hardened oxide or nitride coating of said attachment portion comprises oxidized zirconium, oxidized hafnium, oxidized niobium, oxidized tantalum, nitrided zirconium, nitrided hafnium, nitrided niobium, nitrided tantalum or combinations thereof.

7. The prosthesis of claim 5 wherein said attachment portion is comprised of zirconium or zirconium alloy.

8. The prosthesis of claim 7 wherein said diffusion-hardened oxide or nitride coating comprises blue-black or black oxidized zirconium.

9. The prosthesis of claim 1 wherein said at least one bioceramic compound is selected from the group consisting of hydroxyapatite, fluoroapatite, chloroapatite, bromoapatite, and iodoapatite, calcium sulfate, calcium phosphate, calcium carbonate, calcium tartarate, bioactive glass, and combinations thereof.

10. The prosthesis of claim 9 wherein said compound comprises hydroxyapatite.

11. A prosthesis comprising:
(a) a femoral component having
(1) an implant portion for inserting into body tissue;
(2) a head portion comprising a bearing surface;
said femoral component formed of zirconium, hafnium, niobium, tantalum or alloys thereof;
(b) an acetabular cup having an inner surface comprising an organic polymer or a polymer-based composite and an outer surface, said inner surface being adapted to cooperate with said bearing surface;
(c) a diffusion-hardened oxide or nitride coating on at least a part of said bearing surface for reducing wear of said inner surface; and,
(d) a coating of at least one bioceramic compound on at least a part of:
(1) said implant portion;
(2) said outer surface; or,
(3) both said implant portion and said outer surface.

12. The prosthesis of claim 11 wherein said diffusion-hardened oxide or nitride coating is selected from the group consisting of oxidized zirconium, oxidized hafnium, oxidized niobium, oxidized tantalum, nitrided zirconium, nitrided hafnium, nitrided niobium, nitrided tantalum and combinations thereof.

13. The prosthesis of claim 11 wherein said femoral component is formed of zirconium or zirconium alloy and said diffusion-hardened oxide or nitride coating comprises blue-black or black oxidized zirconium.

14. The prosthesis of claim 11 wherein said outer surface formed of zirconium, hafnium, niobium, tantalum or alloys thereof.

15. The prosthesis of claim 14 wherein at least a part of said outer surface comprises a diffusion-hardened oxide or nitride coating.

16. The prosthesis of claim 15 wherein said diffusion-hardened oxide or nitride coating of said outer surface comprises oxidized zirconium, oxidized hafnium, oxidized niobium, oxidized tantalum, nitrided zirconium, nitrided hafnium, nitrided niobium, nitrided tantalum or combinations thereof.

17. The prosthesis of claim 15 wherein outer surface is comprised of zirconium or zirconium alloy.

18. The prosthesis of claim 17 wherein and said diffusion-hardened oxide or nitride coating comprising said outer surface comprises blue-black or black oxidized zirconium.

19. The prosthesis of claim 11 wherein said at least one bioceramic compound comprises a compound selected from the group consisting of hydroxyapatite, fluoroapatite, chloroapatite, bromoapatite, iodoapatite, calcium sulfate, calcium phosphate, calcium carbonate, calcium tartarate, bioactive glass, and combinations thereof.

20. The prosthesis of claim 19 wherein said compound comprises hydroxyapatite.

21. A prosthesis comprising:
(a) a body having an implant portion for inserting into body tissue, said body formed of zirconium, hafnium, niobium, tantalum or alloys thereof;
(b) a bearing surface on said body, said bearing surface being sized and shaped to engage or cooperate with a second bearing surface, said second bearing surface being a part of another prosthesis portion;
(c) a diffusion-hardened oxide or nitride coating on said bearing surface of said body;
(d) a coating of at least one bioceramic compound on at least a part of said body.

22. The prosthesis of claim 21 wherein said diffusion-hardened oxide or nitride coating is selected from the group consisting of oxidized zirconium, oxidized hafnium, oxidized niobium, oxidized tantalum, nitrided zirconium, nitrided hafnium, nitrided niobium, nitrided tantalum and combinations thereof.

23. The prosthesis of claim 21 wherein said body is formed of zirconium or zirconium alloy and said diffusion-hardened oxide or nitride coating comprises blue-black or black oxidized zirconium.

24. The prosthesis of claim 21 wherein said another prosthesis portion comprises zirconium, hafnium, niobium, tantalum, or alloys thereof.

25. The prosthesis of claim 24 wherein said another prosthesis portion comprises a diffusion-hardened oxide or nitride coating.

26. The prosthesis of claim 25 wherein said diffusion-hardened oxide or nitride coating of said another prosthesis portion comprises oxidized zirconium, oxidized hafnium, oxidized niobium, oxidized tantalum, nitrided zirconium, nitrided hafnium, nitrided niobium, nitrided tantalum or combinations thereof.

27. The prosthesis of claim 26 wherein said another prosthesis portion comprises zirconium or zirconium alloy.

28. The prosthesis of claim 27 wherein and said diffusion-hardened oxide or nitride coating comprising said another prosthesis portion comprises blue-black or black oxidized zirconium.

29. The prosthesis of claim 21 wherein said at least one bioceramic compound comprises a compound selected from the group consisting of hydroxyapatite, fluoroapatite, chloroapatite, bromoapatite, and iodoapatite, calcium sulfate, calcium phosphate, calcium carbonate, calcium tartarate, bioactive glass, and combinations thereof.

30. The prosthesis of claim 29 wherein said compound comprises hydroxyapatite.

31. The prosthesis of claim 21 wherein said another prosthesis portion comprises a coating of at least one bioceramic compound.

32. The prosthesis of claim 32 wherein said coating of at least one bioceramic compound on said another prosthesis portion comprises a compound selected from the group consisting of hydroxyapatite, fluoroapatite, chloroapatite, bromoapatite, and iodoapatite, calcium sulfate, calcium phosphate, calcium carbonate, calcium tartarate, bioactive glass, and combinations thereof.

33. The prosthesis of claim 32 wherein said compound comprises hydroxyapatite.

34. The prosthesis of claim 21 wherein said second bearing surface comprises an organic polymer or polymer composite.

35. A prosthesis comprising:
(a) a body having an implant portion for inserting into the body tissue of a patient, said body formed of zirconium, hafnium, niobium, tantalum or alloys thereof,
(b) a bearing surface on said body;
(c) a counter bearing surface adapted to cooperate with the bearing surface;
(c) a diffusion-hardened oxide or nitride coating at least a part of said bearing surface; and,
(d) a coating of at least one bioceramic compound on at least a part of said implant portion.

36. The prosthesis of claim 35 wherein said diffusion-hardened oxide or nitride coating is selected from the group consisting of oxidized zirconium, oxidized niobium, oxidized hafnium, oxidized tantalum, nitrided zirconium, nitrided niobium, nitrided hafnium, nitrided tantalum and combinations thereof.

37. The prosthesis of claim 35 wherein said body is formed of zirconium or zirconium alloy and said diffusion-hardened oxide or nitride coating comprises blue-black or black oxidized zirconium.

38. The prosthesis of claim 35 wherein said counter bearing surface is comprised of a diffusion-hardened oxide or nitride coating.

39. The prosthesis of claim 38 wherein said diffusion-hardened oxide or nitride coating comprising said counter bearing surface is selected from the group consisting of oxidized zirconium, oxidized niobium, oxidized hafnium, oxidized tantalum, nitrided zirconium, nitrided niobium, nitrided hafnium, nitrided tantalum and combinations thereof.

40. The prosthesis of claim 39 wherein said counter bearing comprises blue-black or black oxidized zirconium.

41. The prosthesis of claim 35 wherein said at least one bioceramic compound comprises a compound selected from the group consisting of hydroxyapatite, fluoroapatite, chloroapatite, bromoapatite, and iodoapatite, calcium sulfate, calcium phosphate, calcium carbonate, calcium tartarate, bioactive glass, and combinations thereof.

42. The prosthesis of claim 41 wherein said compound comprises hydroxyapatite.

43. The prosthesis of claim 35 wherein said counter bearing surface comprises an organic polymer or polymer composite.

44. The prosthesis of claim 35 further comprising an irregular surface formed of beads of zirconium, hafnium, niobium, tantalum, or alloys thereof.

45. The prosthesis of claim 44 further comprising a diffusion-hardened surface on said beads or a coating of at least one bioceramic compound on said beads or both a diffusion-hardened surface and a coating of at least one bioceramic compound on said beads.

46. The prosthesis of claim 35 further comprising an irregular surface formed of wire mesh of zirconium, hafnium, niobium, tantalum, or alloys thereof.

47. The prosthesis of claim 46 further comprising a diffusion-hardened surface on said wire mesh or a coating of at least one bioceramic compound on said wire mesh or both a diffusion-hardened surface and a coating of at least one bioceramic compound on said wire mesh.

48. The prosthesis of claim 35 further comprising a textured surface formed of zirconium, hafnium, niobium, tantalum, or alloys thereof.

49. The prosthesis of claim 48 further comprising a diffusion-hardened surface on said textured surface or a coating of at least one bioceramic compound on said textured surface or both a diffusion-hardened surface and a coating of at least one bioceramic compound on said textured surface.

50. A prosthesis comprising:
(a) a prosthesis body formed of zirconium, hafnium, niobium, tantalum or alloys thereof, the prosthesis body forming one component of a two-component joint and having a bearing surface at least a portion of which is adapted to cooperate with and slide against body tissue of a second joint component;
(b) a diffusion-hardened oxide or nitride coating on at least a part of a bearing surface adapted to cooperate and slide against the body tissue, said coating selected from the group consisting of oxidized zirconium, oxidized hafnium, oxidized niobium, oxidized tantalum, nitrided zirconium, nitrided hafnium, nitrided niobium, nitrided tantalum and combinations thereof; and,
(c) a coating of at least one bioceramic compound on at least a part of said prosthesis body.

51. The prosthesis of claim 50 wherein the bearing surface is a femoral head adapted to cooperate with and slide against cartilage tissue of a pelvis.

52. The prosthesis of claim 50 wherein the bearing surface is a head of a humeral implant adapted to cooperate with natural body tissue of a glenoid of a recipient.

53. The prosthesis of claim 50 wherein the bearing surface is a bearing surface of a glenoid prosthesis adapted to cooperate with natural tissue of a humerus.

54. The prosthesis of claim 50 wherein the bearing surface is a bearing surface of at least one condyle of a femoral component of a knee joint prosthesis adapted to cooperate against natural tissue of a tibia.

55. The prosthesis of claim 50 wherein the bearing surface is a bearing surface of a tibial component of a knee joint prosthesis adapted to cooperate with natural tissue of condyles.

56. The prosthesis of claim 50 wherein said at least one bioceramic compound is a compound selected from the group consisting of hydroxyapatite, fluoroapatite, chloroapatite, bromoapatite, and iodoapatite, calcium sulfate, calcium phosphate, calcium carbonate, calcium tartarate, bioactive glass, and combinations thereof.

57. The prosthesis of claim 50 wherein the prosthesis body formed of zirconium or alloys thereof and the diffusion-hardened oxide or nitride coating comprises blue-black or black oxidized zirconium.

58. A prosthesis comprising:
(a) a body formed of alloy having a composition comprising from about 10 to about 20 wt % niobium or from about 35 to about 50 wt % niobium; from about 13 to about 20 wt % zirconium; and the balance titanium;
(b) a diffusion-hardened oxide or nitride coating on at least a part of said body; and,
(c) a coating of at least one bioceramic compound on at least a part of said body.

59. The prosthesis of claim 58 wherein said composition consists essentially of about 74 wt % titanium, about 13 wt % niobium, and about 13 wt % zirconium.
